(19) United States
(12) Patent Application Publication
(10) Pub. No.: US 2021/0212886 A1
(43) Pub. Date: Jul. 15, 2021

(54) STIMULATION AND TREATMENT DEVICE

(71) Applicant: RODNEY BURNETT-JONES, Ashmore, QLD (AU)

(72) Inventor: Rodney BURNETT-JONES, Ashmore (AU)

(21) Appl. No.: 17/054,098

(22) PCT Filed: May 22, 2019
(86) PCT No.: PCT/AU2019/000062

§ 371 Date: Nov. 9, 2020

(30) Foreign Application Priority Data
May 22, 2018 (AU) 2018901790
Jan. 22, 2019 (AU) 2019900191

(51) Int. Cl. A61H 19/00; A61H 9/00

(52) U.S. Cl. CPC A61H 19/32; A61H 2201/1669; A61H 2201/10; A61H 9/0057

(57) ABSTRACT

A stimulation and treatment device includes an elongate vessel that is closed at a distal end and that defines a vacuum chamber, the vessel including an air extraction passage in fluid communication with the vacuum chamber so that a pump can be connected to the vessel to extract air from the chamber via the air extraction passage. A penile passage member is arranged on a proximal end of the vessel, the penile passage member defining a penile passage through which a penis can extend into the vacuum chamber, the passage member being configured to engage a penis in a substantially sealed manner. An internal slider is arranged on an inner surface of the vessel and is configured for sliding reciprocally within the vessel, the internal slider defining an opening through which the penis can extend and being configured to make contact with the penis to stimulate the penis during reciprocal movement of the internal slider. An external slider is arranged on an outer surface of the vessel and is capable of sliding reciprocal movement along the outside of the vessel, wherein the internal and external sliders are magnetically attracted to each other so that the reciprocal manipulation of the external slider results in a corresponding reciprocal sliding of the internal slider, allowing the penis to be stimulated.

### FIELD OF THE INVENTION

This invention relates to a stimulation and treatment device. More particularly, but not exclusively, the invention relates to a stimulation and treatment device that is suitable for personal stimulation and for the treatment of erectile dysfunction and/or urinary incontinence.

### BACKGROUND OF THE INVENTION

Conventional penis or vacuum pumps are used to engorge a penis while it is located within a pump chamber. Such vacuum pumps enable a user to achieve and maintain an erection within the confines of the pump or vacuum chamber while under vacuum. Conventional vacuum pumps do not allow for direct contact with the penis by hand or by other devices. Thus, manual stimulation is blocked by a wall of the chamber.

Other devices enable the user to stimulate the penis manually using silicone, latex, rubber or other forms of plastic sleeve having fingers, prongs or the like encased in a tube or chamber. A problem with such devices is that they have limited use if the user does not achieve and maintain an erection.

Electrostimulation is known. However, an erection is not guaranteed and contact with the hands is not recommended during electrostimulation.

### SUMMARY OF THE INVENTION

According to one aspect of the invention, there is provided a stimulation and treatment device, which comprises:
an elongate vessel that is closed at one end and that defines a vacuum chamber, the vessel including an air extraction passage in fluid communication with the vacuum chamber so that a pump can be connected to the vessel to extract air from the chamber via the air extraction passage;
a penile passage member arranged on a proximal end of the vessel, the penile passage member defining a penile passage through which a penis can extend into the vacuum chamber, the passage member being configured to engage a penis in a substantially sealed manner;
an internal slider arranged on an inner surface of the vessel and configured for sliding reciprocally within the vessel, the internal slider defining an opening through which the penis can extend and being configured to make contact with the penis to stimulate the penis during reciprocal movement of the internal slider; and
an external slider arranged on an outer surface of the vessel and capable of sliding reciprocal movement along the outside of the vessel, wherein
the internal and external sliders are magnetically attracted to each other so that the reciprocal manipulation of the external slider results in a corresponding reciprocal sliding of the internal slider, allowing the penis to be stimulated.

The elongate vessel may include a tube that is open at both ends and a closure that is detachably mounted on a distal end of the tube to close the distal end.

The closure may be of an elastomeric material such as silicone and may include a seal that engages the distal end of the tube.

The closure may define the air extraction passage. A non-return valve assembly may be positioned in the air extraction passage and may be configured to permit the extraction of air from the chamber.

The closure may include a pump connection port to permit a pump to be connected to the closure for extracting air from the chamber.

The penile passage member may include a fitting that is configured to engage a proximal end of the tube in a substantially sealed, detachable manner, and a penis sealing arrangement positioned in the fitting and defining the penile passage so that the penis can be engaged in a substantially sealed manner by the sealing arrangement.

The penis sealing arrangement may include two rings, at least a portion of each ring being of a conductive material, conductive leads being connected to the conductive material to supply an electrical stimulating signal to the conductive material.

The penile passage member may include an electrostimulation insert that is positioned in the penis sealing arrangement to stimulate the penis, the electrostimulation insert being in conductive contact with the conductive material.

The electrostimulation insert may include two electrostimulation members positioned in spaced, aligned relationship, each electrostimulation member defining a passage through which a penis can extend such that the penis makes electrical contact with the electrostimulation members and each electrostimulation member is in contact with the conductive material of respective rings.

The electrostimulation insert may be positioned in the penis sealing arrangement to make electrical contact with the penis, the electrostimulation insert being connectable to an electrical supply to receive an electrical stimulating signal.

The electrostimulation insert may include two electrostimulation members positioned in spaced, aligned relationship, each electrostimulation member defining a passage through which a penis can extend such that the penis makes electrical contact with the electrostimulation members and each electrostimulation member includes an electrical lead for connection to an electrical supply.

Each electrostimulation member includes a body with a series of projections that extend into the passage from an inner surface of the body, the projections being positioned to make electrical contact with the penis when the penis is received in the passage.

Each electrostimulation member may be of a resiliently flexible material.

A connector may be arranged on the electrical lead, the connector being configured so that a transcutaneous electrical nerve stimulation machine can be connected to the lead.

The internal slider may include an annular inner slide and may be dimensioned to make sliding contact with the inner surface of the tube.

An annular stimulation insert may be mounted on the inner slide, the stimulation insert defining the opening through which the penis can extend.

The stimulation insert may be in the form of a material suitable for reciprocal stimulation, such as a hypoallergenic, elastomeric material.

The inner slide may include a body with at least one magnet in or on the body.

The external slider may include an annular outer slide dimensioned to make sliding contact with the outer surface of the tube. The outer slide may include a body with at least one magnet in the body.

According to another aspect of the invention, there is provided an electrostimulation device, or member, the device comprising:
a body that defines a passage for receiving a penis; and
at least one penis contacting formation on or in the body for making conductive contact with the penis, at least the penis contacting formation being of an electrically conductive material, wherein
at least the penis contacting formation is connectable to an electrical stimulating signal.

### BRIEF DESCRIPTION OF THE DRAWINGS

FIG. 1 shows an exploded, side sectioned view of one embodiment of a stimulation and treatment device, in accordance with the invention, in a partially disassembled configuration.

FIG. 2 shows the embodiment of FIG. 1, in an assembled configuration.

FIG. 3 shows a distal, three-dimensional view of a penile passage member of the device of FIG. 1.

FIG. 4 shows a distal view of the penile passage member of FIG. 3.

FIG. 5 shows a schematic sectioned side view of the penile passage member of FIG. 4.

FIG. 6 shows a proximal three-dimensional view of a closure of the device of FIG. 1.

FIG. 7 shows a schematic sectioned side view of the closure of FIG. 6.

FIG. 8 shows a proximal side of the closure of FIG. 6.

FIG. 9 shows a three-dimensional view of an internal slider of the device of FIG. 1.

FIGS. 10a to 10e show various stages of assembly of a vacuum tube and internal and external sliders of the device of FIG. 1 or FIG. 2.

FIG. 11 shows a three-dimensional view, from a proximal end, of a further embodiment of a stimulation and treatment device, in accordance with the invention.

FIG. 12 shows a three-dimensional view, from a distal end, of the stimulation and treatment device of FIG. 11.

FIG. 13 shows a distal, three-dimensional view of a penile passage member, internal and external sliders and a cap of the device of FIG. 11 in a disassembled condition.

FIG. 14 shows a three-dimensional view of a tube of the device of FIG. 11.

FIG. 15 shows a three-dimensional view of a stimulation insert for the internal slider of the device of FIG. 11.

FIG. 16 shows an assembled part of the penile passage member, including electrostimulation members, of the device of FIG. 11.

FIG. 17 shows a distal, three-dimensional view of the penile passage member of the device of FIG. 11, in a disassembled configuration, without an electrostimulation member.

FIG. 18 shows the penile passage member of FIG. 17 in an assembled configuration.

FIG. 19 shows a distal, three-dimensional view of part of the penile passage member of FIG. 17 showing an electrostimulation member.

FIG. 20 shows a distal end view of the electrostimulation member in a distal ring of the penile passage member.

FIG. 21 shows a proximal end view of part of the penile passage member of the device of FIG. 11, including an electrostimulation member.

FIG. 22 shows a proximal end view of part of the penile passage member of FIG. 17.

FIG. 23 shows a distal end view of the part of the penile passage member of FIG. 22.

FIG. 24 shows a schematic sectioned side view of an assembled penile passage member of FIG. 22.

FIG. 25 shows a disassembled view of the internal slider of the device of FIG. 11.

FIG. 26 shows an exploded view of the external slider, the internal slider and a stimulation insert for the internal slider.

FIG. 27 shows the external and internal sliders out of alignment with each other.

FIG. 28 shows the external and internal sliders in alignment with each other.

FIG. 29 shows a proximal, three-dimensional view of a further embodiment of a stimulation and treatment device.

FIG. 30 shows a distal, three-dimensional view of the device of FIG. 29.

FIG. 31 shows a distal three-dimensional view of a penile passage member, internal and external sliders and a closure of the device of FIG. 29 in a disassembled configuration.

FIG. 32 shows a distal three-dimensional view of a tube of the device of FIG. 29.

FIG. 33 shows a three-dimensional view of a stimulation insert for the internal slider of the device of FIG. 29.

FIG. 34 shows a schematic sectioned side view of the device of FIG. 29.

FIG. 35 shows a proximal, three-dimensional, disassembled view of a penile passage member of the device of FIG. 29.

FIG. 36 shows a distal, three-dimensional, disassembled view of the penile passage member of FIG. 35.

FIG. 37 shows a distal, three-dimensional view of the penile passage member of FIG. 35.

FIG. 38 shows an electrostimulation member for the penile passage member of either of the embodiments of the stimulation and treatment device described herein.

FIG. 39 shows a schematic sectioned side view of an assembled penile passage member incorporating electrostimulation members, for either of the embodiments of the stimulation and treatment device described herein.

FIG. 40 shows a distal, three-dimensional view of a closure of the device of FIG. 29.

FIG. 41 shows a proximal, three-dimensional view of the closure of FIG. 40.

FIG. 42 shows a schematic side sectioned view of a further embodiment of a stimulation and treatment device, in accordance with the invention.

FIG. 43 shows a disassembled internal slider and insert and external slider of the device of FIG. 42.

FIG. 44 shows the internal and external sliders of the device of FIG. 43 out of alignment with each other.

FIG. 45 shows the internal and external sliders of the device of FIG. 43 in alignment with each other.

FIG. 46 shows a further embodiment of a stimulation and treatment device, in accordance with the invention

### DETAILED DESCRIPTION

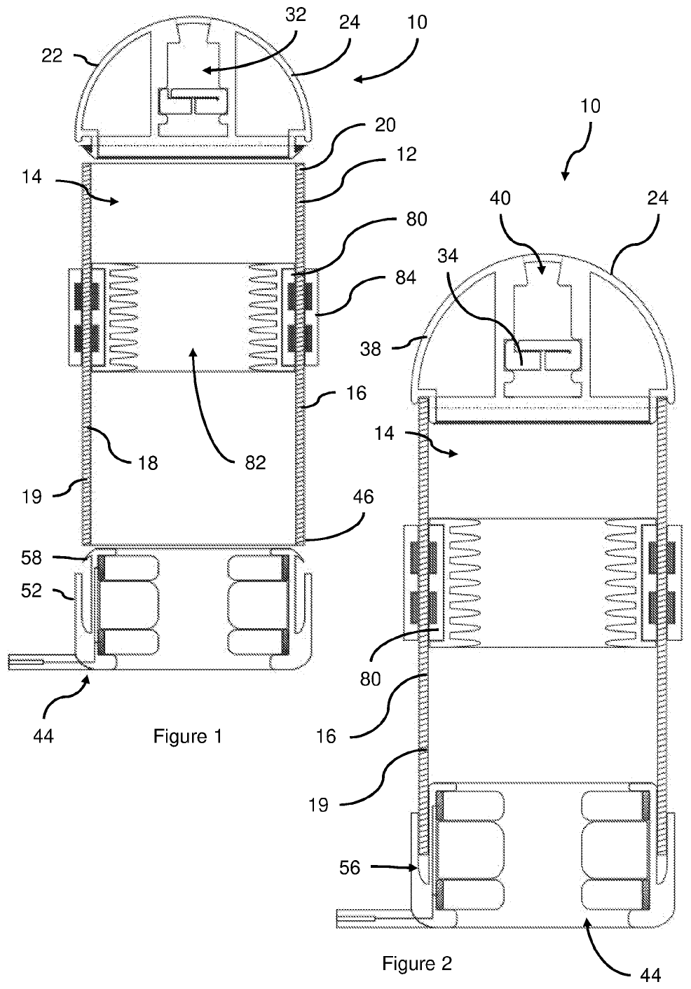

In FIGS. 1 and 2, reference numeral 10 generally indicates one embodiment of a stimulation and treatment device, in accordance with the invention. The device 10 is suited for personal stimulation of the penis or for the treatment of erectile dysfunction and/or urinary incontinence.

The device 10 includes an elongate vessel 12 that defines a vacuum chamber 14 and is closed at one end. The vessel 12 includes a tube 16 that is open at both ends. The tube 16 is circular cylindrical and can be of any one of a number of materials, including a plastics material, rubber or a metal. Instead of being circular cylindrical, the tube 16 can have an oval cross-section or some other cross-sectional, multi-sided shape, such as hexagonal, depending on requirements. An inner surface 18 of the tube 16 is smooth to facilitate sliding engagement with an internal slider, described in further detail below. The inner surface 18 can be textured or can be otherwise configured to facilitate sliding. For example, the inner surface 18 can define ridges or other formations that reduce frictional contact with the internal slider. An outer surface 19 of the tube 16 is also smooth to facilitate sliding displacement of an external slider relative to the tube 16. The outer surface 19 could also be textured, depending on technical and commercial requirements. As with the inner surface 18, the outer surface 19 could also define ridges or other formations that reduce frictional contact with the external slider. They can also be configured to be aesthetically pleasing. The tube 16 can be transparent or opaque and can also be coloured.

A closure 22 is mounted on a distal end 20 of the tube 16 to engage the distal end 20 in a sealed manner. The closure 22 includes a cap 24 of an elastomeric material, such as silicone. The cap 24 can be of other suitable materials, including latex, rubber or a different plastics material.

It is to be appreciated that the closure 22 can form part of the vessel 12, so closing the vessel.

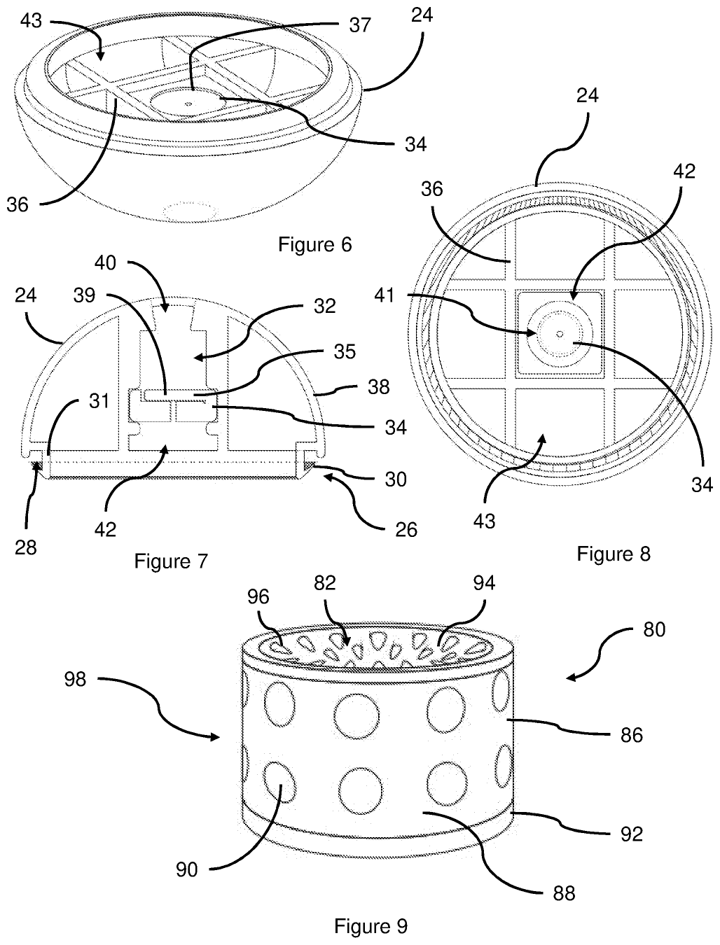

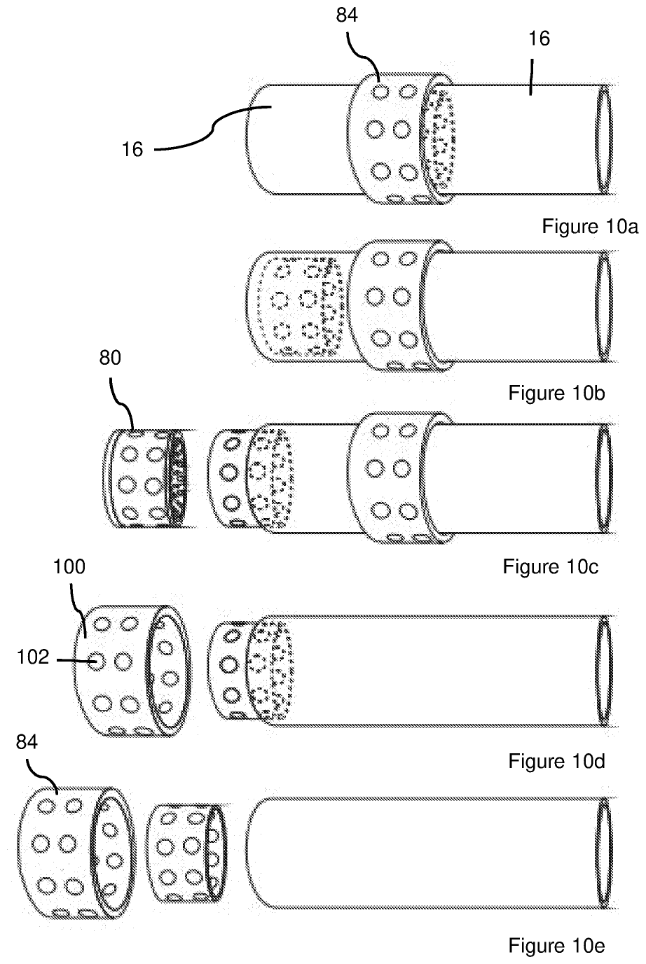

Further detail of the cap 24 is shown in FIGS. 6 to 8. The cap 24 has a domed or part-spherical profile. A distal, peripheral sealing formation 26 extends from the cap 24 and is shaped to engage the distal end 20 of the tube 16 in a detachable, sealed manner. To that end, the sealing formation 26 defines a peripheral channel 28. The channel 28 is dimensioned so that the distal end 20 can be received in the channel 28. A lip 30 extends from an inner wall 31 of the channel 28, distally and into the channel 28. Thus, when a vacuum is generated within the chamber 14, the ingress of air is inhibited as a result of radial pressure being exerted on the lip 30 causing the lip 30 to bear against the outer surface 19 of the tube 16, enhancing a sealing engagement of the cap 24 with the tube 16.

The cap 24 defines an air extraction passage 32 in fluid communication with the vacuum chamber 14. A non-return valve assembly 34 is arranged in the passage 32 such that air can be extracted from the chamber 14, in a non-return manner.

Baffles 36 extend from an internal surface of a wall 38 of the cap 24. The baffles 36 define a central receptacle 42 in which the valve assembly 34 is located. The cap 24 defines a vacuum pump connection port 40 in fluid communication with the valve assembly 34. The central receptacle 42 opens into the chamber 14 so that the connection port 40, the valve assembly 34 and the central receptacle 42 define the air extraction passage 32. The connection port 40 can be connected to a conventional vacuum pump of the type used with current penis pumps. Such a vacuum pump can be manually operated or can be an electrical pump which is remotely operated by a user. It will be appreciated that the cap 24 may be provided without the baffles 36.

The valve assembly 34 includes a nonreturn valve 35 that is seated within the central receptacle 42. The nonreturn valve 35 includes a valve body 37 that defines a valve passage 41 through which the air can move. A valve closure 39 is pivotally mounted on the valve body 37, to open when air is extracted from the chamber 14 and to close against the ingress of air into the chamber 14. The valve body 37 and the valve closure 39 can be of a one-piece moulding. The moulding can be of an elastomeric material, or some other suitable material.

The baffles 36 also define fluid collection receptacles 43, to collect ejaculate and urine, if necessary.

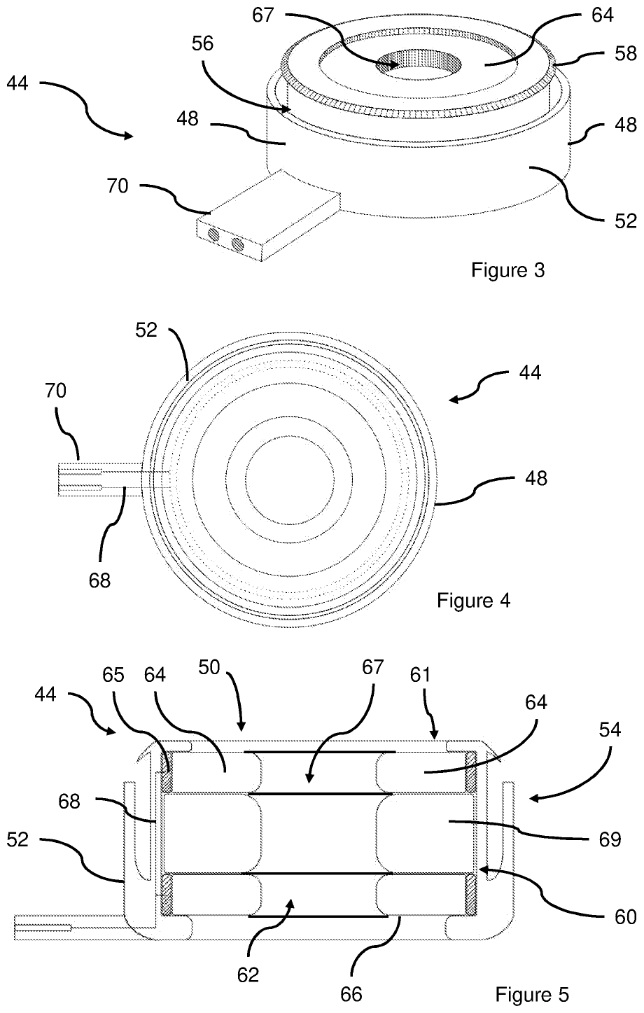

A penile passage member in the form of a penile aperture assembly 44 is mounted on a proximal end 46 of the tube 16. The aperture assembly 44 is configured to engage the penis in a substantially airtight manner. Detail of the penile aperture assembly 44 is shown in FIGS. 3 to 5.

The aperture assembly 44 includes a fitting 48. The fitting 48 can be of an elastomeric material, such as silicone. The fitting 48 has a wall 52 that defines a passage 50. A peripheral sealing formation 54 is arranged on the wall 52 and is shaped to engage the proximal end 46 of the tube 16 in a detachable, sealed manner. The wall 52 defines a peripheral channel 56 in which the proximal end 46 of the tube 16 can be received. A lip 58 extends from an inner wall of the channel 54, distally and into the channel 56. Thus, when a vacuum is generated within the chamber 14, the ingress of air is inhibited as a result of radial pressure being exerted on the lip 58.

The wall 52 defines an annular, inwardly opening recess or channel 60. A penis sealing arrangement 62 is arranged in the channel 60 and is configured to engage the penis in a substantially sealing manner. The sealing arrangement 62 includes one or more rings of a hypoallergenic, elastomeric material. The one or more rings can include at least one ring of a hypoallergenic, conductive, elastomeric material. More particularly, the one or more rings can include a distal conductive ring 64 and a proximal conductive ring 66, with a sealing ring 69 interposed between the distal and proximal rings 64, 66. The elastomeric material can be in the form of latex or some other suitable material. The rings 64, 66 can be made from conductive silicone. Alternatively, they can be made of other conductive materials, including metals. The channel 60 can be dimensioned to allow for different sized rings 64, 66 to be used depending on individual requirements. The rings 64, 66 can have various internal configurations. Each ring 64, 66 is an electrostimulation member and can take the form or either of the electrostimulation members described below, for example.

A conductive wire 68 can be connected to each of the rings 64, 66, via conductive insets 65, and to an electrical connector 70. The electrical connector 70 can be connected to an electrical stimulation device, such as a TENS (transcutaneous electrical nerve stimulation) machine. Thus, the rings 64, 66 can form an electrostimulation insert 61. Conventionally, TENS machines are used for treating pain and are usually connected to the skin using two or more electrodes. A typical battery-operated TENS unit can modulate pulse width, frequency and intensity. Thus, in this embodiment, the electrostimulation component of the TENS unit is built into the penile aperture assembly 44. It is envisaged that the electrostimulation component can be attached to other components of the device 10. It follows that the TENS unit can be a conventional TENS unit that is adjustable to provide an appropriate stimulation signal.

The rings 64, 66 and the sealing ring 69 define a penile passage 67 into which the penis can extend. The dimensions and material of the rings 64, 66 and the sealing ring 69 can be selected so that the passage can accommodate differently sized penises.

The device 10 includes an internal slider 80 that is arranged on the inner surface 18 of the vessel 12 and is configured for sliding reciprocally within the vessel 12. The internal slider 80 defines an opening 82 through which the penis can extend. The internal slider 80 is configured to make sliding contact with the penis such that reciprocal movement of the internal slider 80 can stimulate the penis.

The device 10 includes an external slider 84 that is arranged on the outer surface 19 of the vessel 12 and is configured for sliding reciprocally over the outer surface 19.

The internal and external sliders 80, 84 are magnetically attracted to each other so that reciprocal manipulation of the external slider 84 results in a corresponding reciprocal sliding of the internal slider 80, allowing the penis to be stimulated.

In addition to FIGS. 1 and 2, further detail of the internal slider 80 can be seen in FIG. 9. The internal slider 80 includes an annular inner slide 86. The slide 86 includes an annular body 88. The body 88 can be of a relatively rigid plastics material, or some other suitable material which is non-metallic. A number of magnets 90 can be embedded or mounted in the body 88. The magnets 90 can be rare earth or neodymium magnets. Instead of separate magnets, the body 88 can be of a magnetised material. Various other forms of magnetic arrangements are envisaged, since it is possible to fabricate magnetic material of various shapes.

A stimulation insert 92 is mounted in the slide 86. The insert 92 is of a hypoallergenic, elastomeric material suitable for skin contact. The material can be silicone or latex, for example. The insert 92 defines the opening 82 through which the penis can extend. The insert 92 can have an inner surface 94 with a plurality of stimulating formations 96 extending from the inner surface 94 to engage the penis in a stimulating manner. The formations 96 can take various configurations.

The insert 92 defines an external annular recess 98 in which the slide 86 can be received. Thus, in use, the insert 92 can be removed and replaced.

The external slider 84 includes an annular body 100. The body 100 can be of a relatively rigid plastics material, or some other suitable material which is non-metallic. A number of magnets 102 can be embedded or mounted in the body 100. The magnets 102 can be rare earth or neodymium magnets. The magnets 90 and the magnets 102 can be arranged with opposite polarity so that they attract each other. Instead of separate magnets, the body 100 can be of a magnetised material. Various other forms of magnetic arrangements are envisaged, since it is possible to fabricate magnetic material of various shapes.

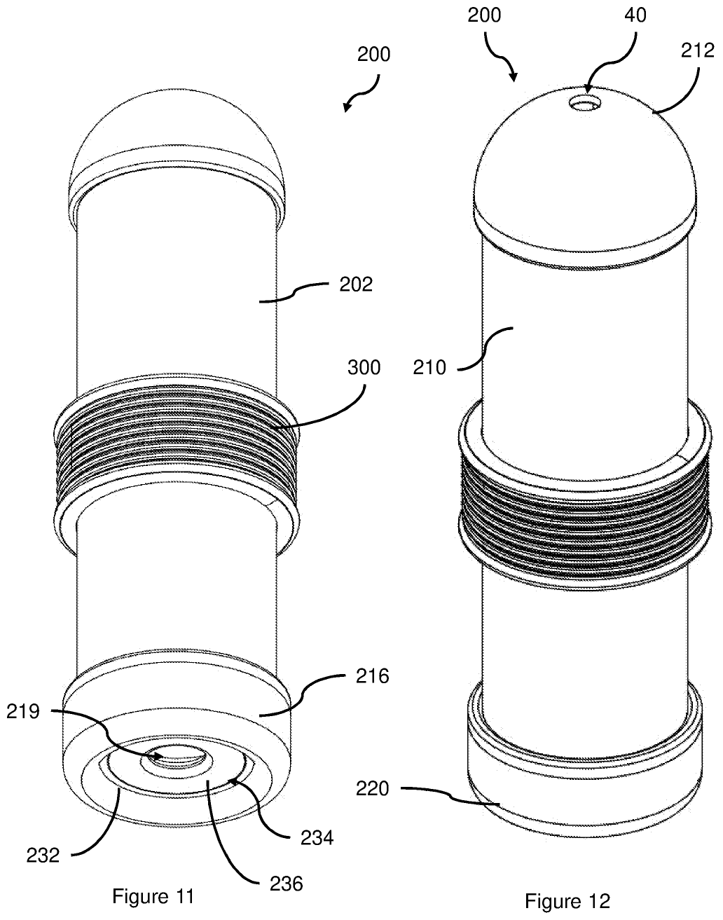

In FIGS. 11 and 12, reference numeral 200 generally indicates one embodiment of a stimulation and treatment device, in accordance with the invention.

The device 200 includes an elongate vessel 202 that defines a vacuum chamber 204. The vessel 202 includes a tube 206 that is open at both ends. The tube 206 is circular cylindrical and can be of any one of a number of materials, including a plastics material, rubber or metal. Instead of being circular cylindrical, the tube 206 can have an oval cross-section or some other cross-sectional, multisided shape, such as hexagonal, depending on requirements. An inner surface 208 of the tube 206 is smooth to facilitate sliding engagement with an internal slider, described in further detail below. The inner surface 208 can also be textured or otherwise configured to facilitate sliding of the internal slider relative to the inner surface 208. For example, the inner surface 208 can define ridges or other formations to minimise frictional contact between the inner surface 208 and the internal slider. An outer surface 210 of the tube 206 is also smooth to facilitate sliding displacement of an external slider relative to the tube 206. However, the outer surface 210 could also be textured, depending on technical and commercial requirements. The outer surface 210 could also define ridges or other formations to minimise frictional contact between the outer surface 210 and the external slider.

A closure 212 is mounted on a distal end 214 of the tube 206 to engage the distal end 214 in a sealed manner. The closure 212 is of an elastomeric material, such as silicone. The closure 212 can be of other suitable materials, including latex, rubber or a different plastics material. The closure 212 can be similar to the closure 22 described above.

A penile passage member in the form of a penile aperture assembly 216 is mounted on a proximal end 218 of the tube 206. The penile aperture assembly 216 is configured to engage a penis in a substantially sealed manner. The penile aperture assembly 216 includes an end fitting 220 that engages the proximal end 218 of the tube 206. The end fitting 220 includes a sidewall 222. The sidewall 222 includes an outer wall portion 226 and an inner wall portion 228, spaced from the outer wall portion to define a distally opening channel 224, between the wall portions 226, 228, for receiving the proximal end 218 of the tube 206, as a snug fit.

The inner wall portion 228 has a distal end portion 230 that defines a proximal facing channel 231 (FIG. 24).

The end fitting 220 has a floor 232 that defines an aperture 234.

The end fitting 220 is of a resiliently flexible material. For example, the end fitting 220 is of an elastomeric material. More particularly, the end fitting 220 can be of a hypoallergenic, elastomeric material.

The penile aperture assembly 216 includes a penis sealing arrangement 217 positioned in the fitting 220 and defining an aperture or penile passage 219 (FIG. 18) configured for sealing engagement with the penis. The sealing arrangement 217 includes a proximal penile aperture member 236 that is engageable with the floor 232 to be received in the aperture 234 and retained therein as a result of the resilient flexibility of the end fitting 220. The aperture member 236 can be glued or welded in position. To that end, the floor 232 defines a shoulder 221 (FIG. 24) to receive the proximal penile aperture member 236.

In FIG. 17, an exploded view of the penile aperture assembly 216 is shown without electrostimulation members 254, described below, which are optional.

The internal wall portion 228 defines an annular recess 238. The proximal penile aperture member 236 is received within the inner wall portion 228.

The assembly 216 further includes a proximal ring 242. The proximal ring 242 is positioned within the recess 238. The proximal ring 242 is of a relatively rigid plastics material. The proximal ring 242 has a proximal conductive portion 243 and a distal non-conductive portion 245.

A proximal electrostimulation device or member 254.1 (FIG. 24) is positioned within the proximal ring 242 and is in conductive abutment with the conductive portion 243 of the proximal ring 242.

The assembly 216 also includes a distal ring 246 that is received within the inner wall portion 228. A proximal end of the distal ring 246 defines an inwardly stepped region 252 so that the distal sealing ring 246 can be brought into nesting engagement with the proximal sealing ring 242.

A distal end portion of the distal ring 246 is outwardly stepped to define a shoulder on which a distal penile aperture member 240 is positioned within the inner wall portion 228 so that the proximal conductive insert 254.1 is interposed between the penile aperture members 236, 240. The distal penile aperture member 240 can be glued, welded or pressed into position.

The penile aperture members 236, 240 are of an annular, disc -shaped configuration. The members 236, 240 define apertures 237, 241, respectively. The apertures 237, 241 are dimensioned, and a material of the members 236, 240 is selected so that the apertures 237, 241 can be brought into sealing engagement with a penis. More particularly, the material is selected to be resiliently flexible to accommodate differently sized penises. The material can be of a suitable hypoallergenic, elastomeric material. An example of such a material is hypoallergenic silicone. As can be seen in the drawings, structural integrity and stability of the members 236, 240 are maintained as a result of the positioning of the members 236 within the fitting 220 and their relationship with the proximal and distal rings 242, 246. This allows the device 200 to be used without the electrostimulation members. Alternatively, the electrostimulation members can simply be disconnected when not required.

A distal edge of the distal ring 246 is positioned in the proximal facing channel 231 to locate the distal ring 246 in the fitting 220.

The distal ring 246 is also of a relatively rigid plastics material. The distal ring 246 has a proximal non-conductive portion 247 and a distal conductive portion 249.

A proximal electrostimulation member 254.1 is positioned in the inner wall portion 228 to be in conductive contact with the conductive portion 243 of the proximal ring 242 (FIG. 24). A distal electrostimulation member 253 is positioned in the inner wall portion 228 to be in conductive contact with the conductive portion 249 of the distal ring 246. Thus, the proximal and distal electrostimulation members 254.1, 254.2 form an electrostimulation insert 255 for the penile aperture assembly 216.

Conductive connectors 257 are positioned in the outer wall portion 226. The conductive connectors 257 can be configured for connection to the TENS machine referred to above. Suitable conductive wires 259 can interconnect the connectors 257 and the respective electrostimulation members 254.1, 254.2 Thus, a suitable stimulatory signal can be provided to the electrostimulation members 254.1, 254.2 with a transcutaneous electrical nerve stimulation (TENS) machine described in further detail below.

The electrostimulation members 254.1, 254.2 are of conductive silicone. The electrostimulation members 254.1, 254.2 have an annular body 256 with an outer surface coated with a nonconductive silicone layer. A series of fingers or ribs 258 project radially inwardly from an inner surface of the body 256. The body 256 defines a passage 261 through which a penis can extend so that the penis can be stimulated by the ribs 258. Other suitable materials may be appropriate for the electrostimulation members. For example, other conductive, hypoallergenic materials may be suitable. In addition, instead of the fingers or ribs 258, other forms of projections could extend from the inner surface of the body 256.

It is envisaged that the electrostimulation members 254, or their equivalents, could be supplied separately to the stimulation and treatment device described herein. Thus, the electrostimulation members could be used separately of the device by simply connecting them to a suitable machine such as the TENS machine referred to herein.

In that case, the annular body 256 could take other forms. For example, the annular body 256 could have a toroidal shape or any other shape that might be suitable for location on a penis.

The component shown in FIG. 19, including the distal ring 246 and electrostimulation member 254.2 can be interchanged with a one-piece, electrostimulation member having a similar shape. In that case, the electrostimulation member is substantially entirely of a conductive material and can be separated from the electrostimulation member 254.1 by a suitable seal of a nonconductive material to avoid shorting between the member 254.1 and the alternative electrostimulation member.

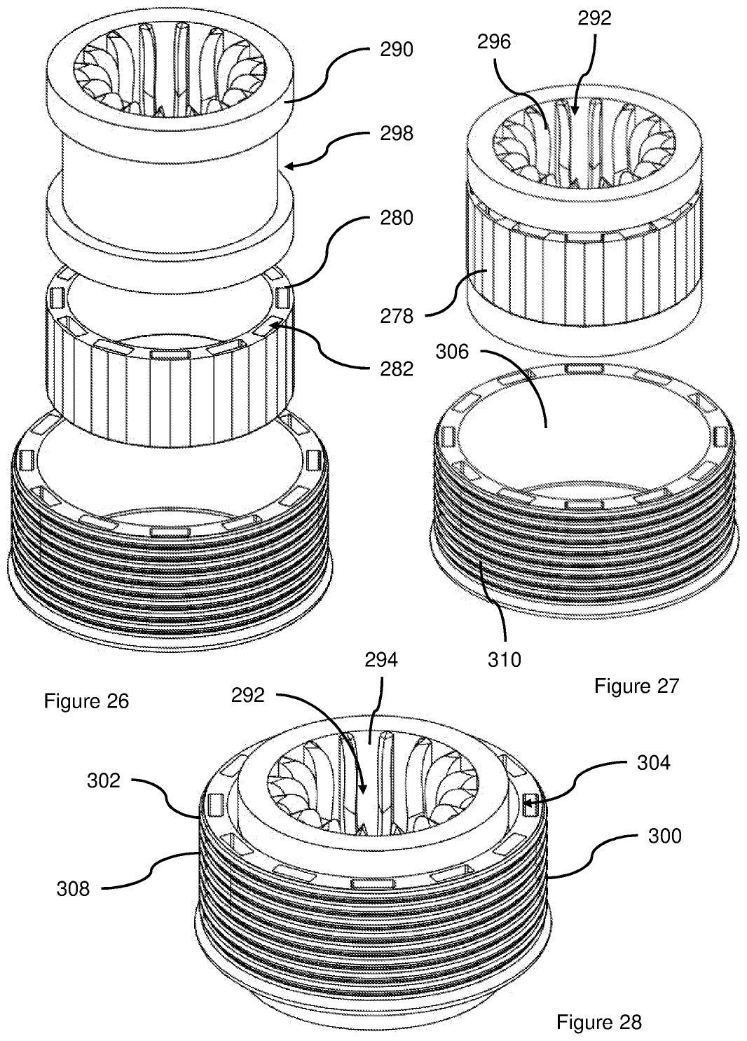

The stimulation and treatment device 200 includes an internal slider 276, details of which are shown in FIGS. 25 to 28. The internal slider 276 includes an annular inner slide 278. The slide 278 includes an annular body 280. The body 280 can be of a relatively rigid plastics material, or some other suitable material which is non-metallic. The body 280 defines a plurality of longitudinally extending sockets 282. The sockets 282 are evenly spaced about a circumference of the body 280. An elongate magnet 284 is positioned in each socket 282. An annular end closure 286 has a series of projections 288 so that the closure 286 can be fitted to the body 282 to close the sockets 282 and so retain the magnets 284 in the sockets 282. It is envisaged that other forms of magnetisation of the internal slider 276 might be appropriate. For example, the magnets 284 may be permanently embedded in the slider 276. Alternatively, the body 280 may itself be magnetised.

The magnets 284 may be rare earth magnets. The magnets 284 may be of neodymium.

The device 200 includes a stimulation insert 290 that is mounted in the slide 278. The insert 290 is of a resiliently flexible material. The material can be a hypoallergenic, elastomeric material suitable for skin contact. The material can be silicone or latex, for example. The insert 290 defines an opening or passage 292 through which the penis can extend.

The insert 290 has an inner surface 294 with a plurality of stimulating formations 296 extending into the passage 292 to engage the penis in a stimulating manner when the slider 276 is moved reciprocally within the tube 206. The formations 296 can take different forms. These can vary from fins to nodules, for example.

The insert 290 defines an annular recess 298 in which the slide 278 can be received. The elastomeric nature of the member 290 allows removal and replacement of the insert 290 in the recess 298.

As can be seen in FIGS. 27 and 28, the inner slide 278 is proud of the stimulation insert 290. The inner slide 278 is dimensioned so that it can engage the inner surface 208 of the tube 206 in a sliding manner. Thus, the stimulation insert 290 is kept spaced from the inner surface 208 so as not to interfere with the sliding of the inner slider 278 relative to the tube 206.

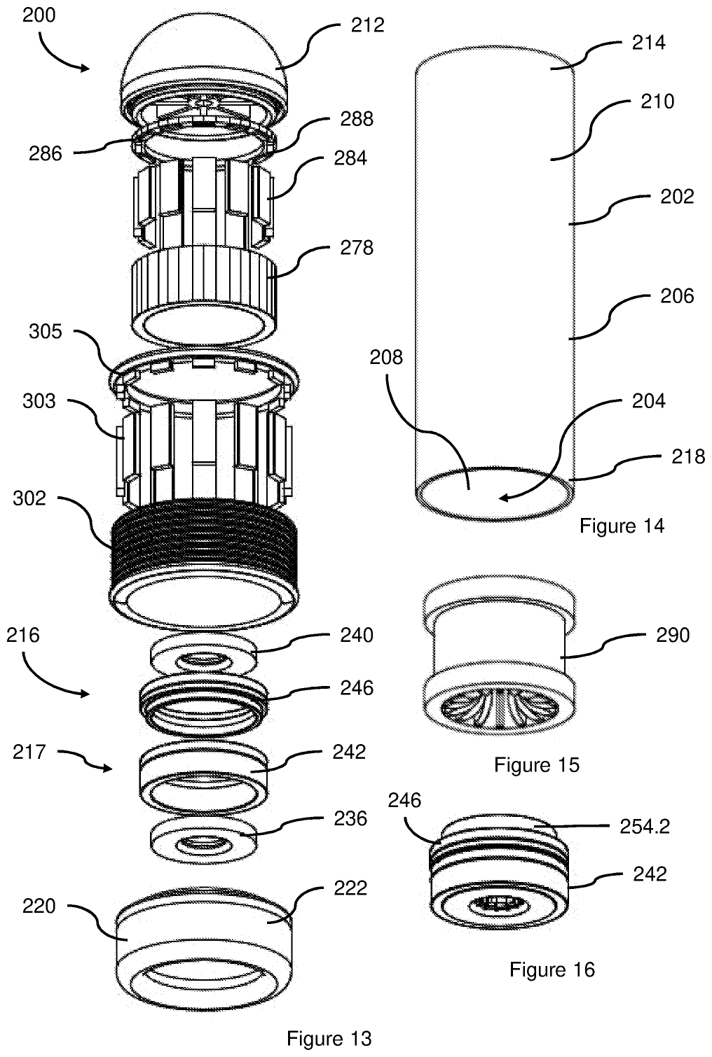

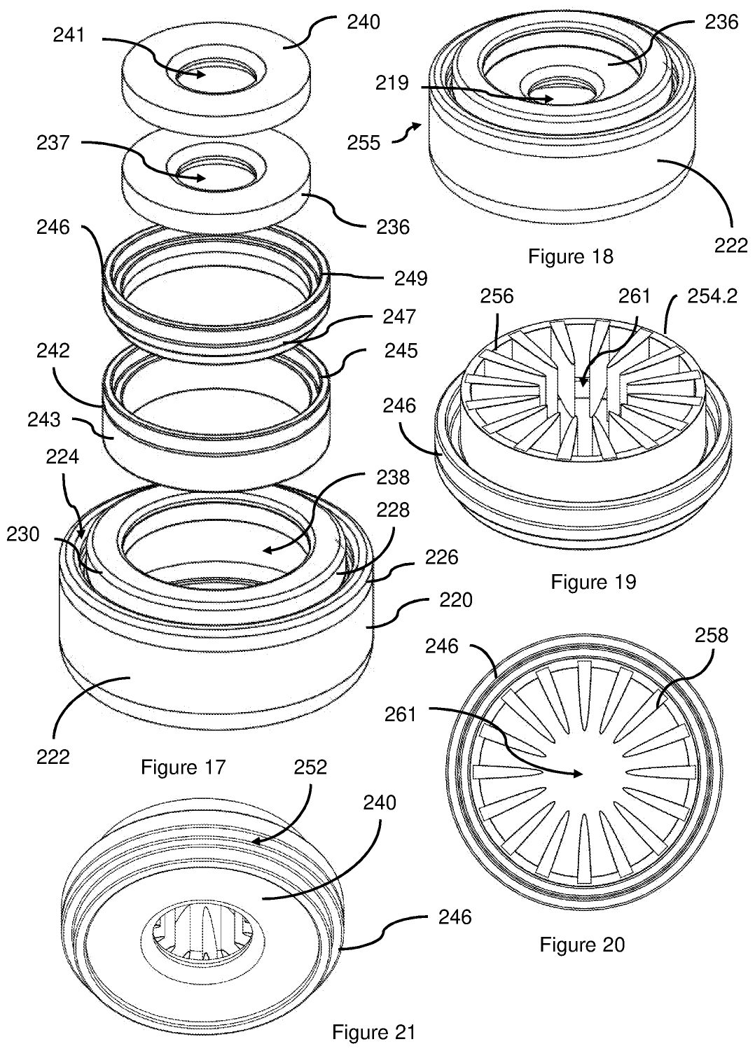

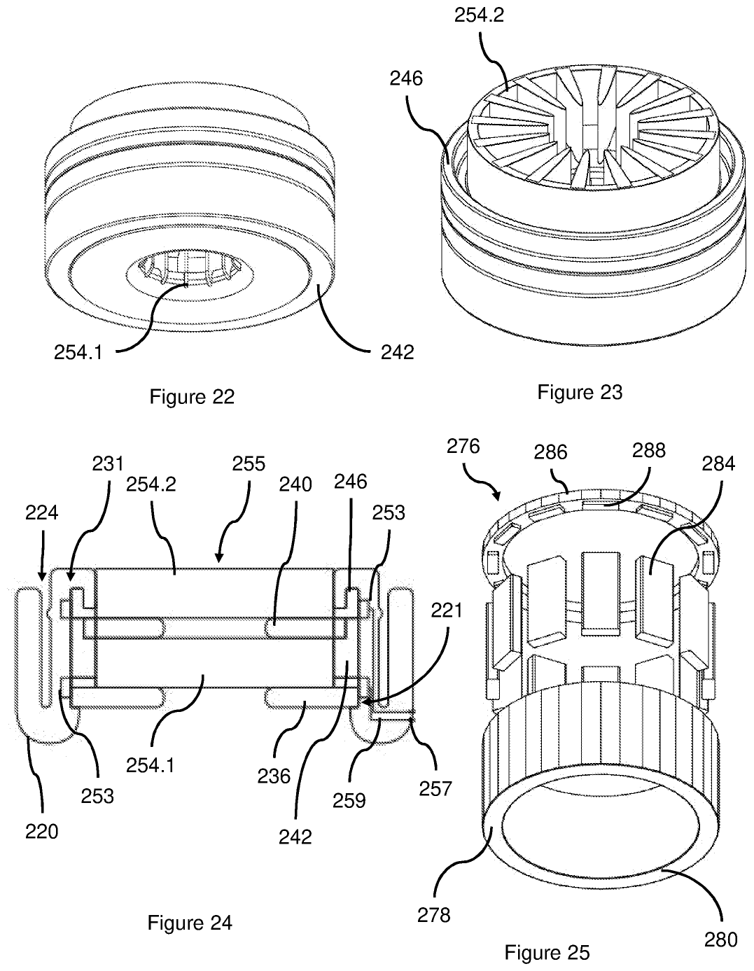

The device 200 includes an external slider 300. The external slider 300 includes an annular body 302. The body 302 can be of a relatively rigid plastics material, or some other suitable material which is non-metallic. The body 302 defines a plurality of longitudinally extending sockets 304. The sockets 304 are evenly spaced about a circumference of the body 302. An elongate magnet 303 (FIG. 13) is positioned in each socket 304. An annular end closure 305 similar to the end closure 286, can have a series of projections so that the closure 305 can be fitted to the body 302 to retain the magnets 303 in the sockets 304.

The body 302 is dimensioned so that an internal surface 306 is configured for sliding engagement with the external surface of the tube 206. The external surface is suitably textured to accommodate such sliding movement.

An external surface 308 of the slider 300 has a series of ribs or projections 310 to facilitate gripping of the slider 300. Instead, the external surface 308 can be textured or profiled to facilitate gripping. The magnets 284 in the internal slider 276 are oriented relative to the magnets in the external slider 300 such that the sliders 276, 300 are attracted to each other. Thus, reciprocal movement of the external slider 300 by a user results in corresponding reciprocal movement of the internal slider. As a result, a user can self-stimulate when the penis is received in the passage 292.

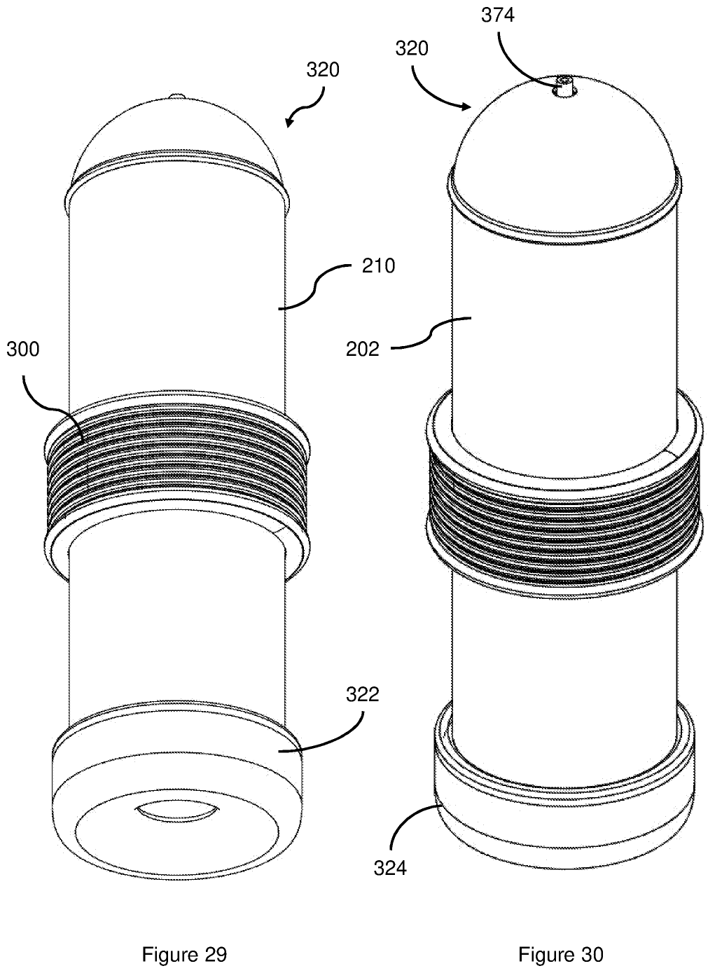

In FIGS. 29 and 30, reference numeral 320 generally indicates a further embodiment of a stimulation and treatment device, in accordance with the invention. With reference to the preceding drawings, like reference numerals refer to like parts, unless otherwise specified.

The device 320 includes a penile passage member in the form of a penile aperture assembly 322 that is somewhat different to the aperture assembly 216 of the device 200.

The aperture assembly 322 includes an end fitting 324. The end fitting 324 includes a sidewall 326 (FIG. 34) that extends peripherally from a floor 328. The floor 328 defines a peripheral recess 330. A peripheral lip 332 extends radially inwardly from a distal end of the sidewall 326. An annular sealing ring 334 is located within the sidewall 326 and is seated in the peripheral recess 330. The annular sealing ring includes an internal wall portion 336 and an external wall portion 338, such that a channel 340 is defined between the wall portions 336, 338.

An end cap 342 includes an end plate 344 that defines a penile aperture 346. The end plate 344 has a similar configuration to the penile aperture members 236, 240.

A cylindrical wall 348 depends from the end plate 344 and is received within the internal wall portion 336 of the sealing ring 334. The end plate 344 overhangs the wall portion 336. When the components are assembled, an internal peripheral edge of the lip 332 and an external peripheral edge of the end plate 344 are in register with the channel 340. The proximal end of the tube 206 can be press fitted into the channel 340.

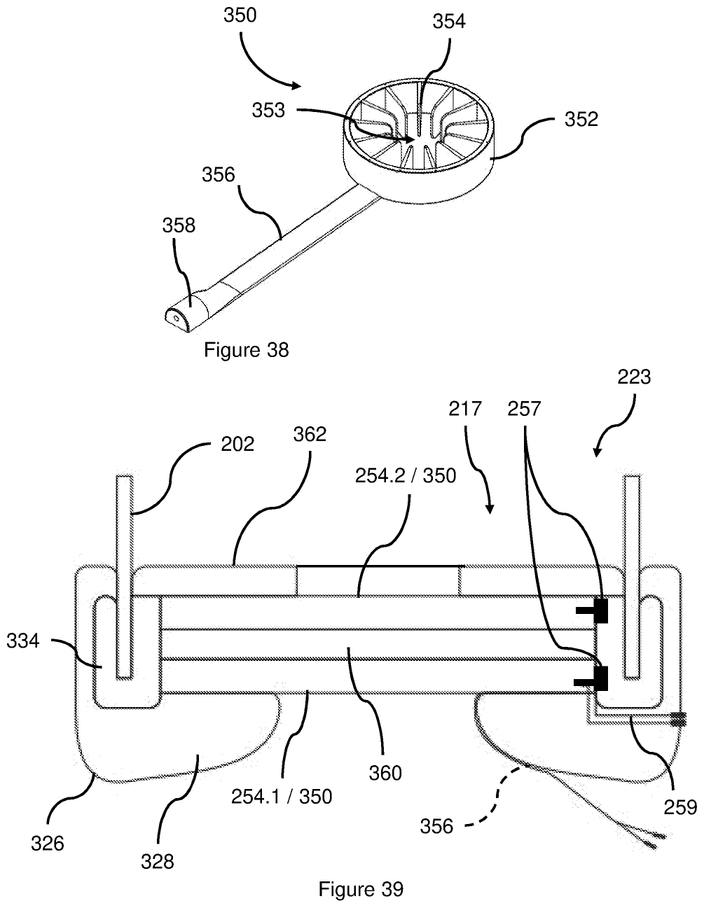

In FIG. 38, there is shown an electrostimulation member 350 for the penile aperture assembly 322. The electrostimulation member 350 is of conductive silicone. The member 350 has an annular body 352 with an outer surface coated with a non-conductive silicone layer. A series of fingers or ribs 354 extend radially inwardly from an inner surface of the body 352. The body 350 defines a passage 353 through which a penis can extend so that the penis can be stimulated by the ribs 354. Other suitable materials may be appropriate for the electrostimulation member. For example, other conductive, hypoallergenic materials may be suitable. In addition, instead of the fingers or ribs 354, other forms of projections could extend from the inner surface of the body 352.

An elongate electrical lead 356 extends from the body 352. The lead 356 is also of a conductive material, in this example, conductive silicone. The lead 356 is coated with a non-conductive silicone layer. The lead 356 includes an electrical connector 358. The electrical connector 358 is configured so that a lead from a TENS machine can be connected to the lead 356 to supply the ribs 354 with electro-stimulation impulses.

It is envisaged that the electrostimulation member 350, or its equivalents, can be supplied separately, in pairs, to the stimulation and treatment device described herein. Thus, the electrostimulation members could be used separately of the device by simply connecting them to a suitable machine such as the TENS machine referred to herein.

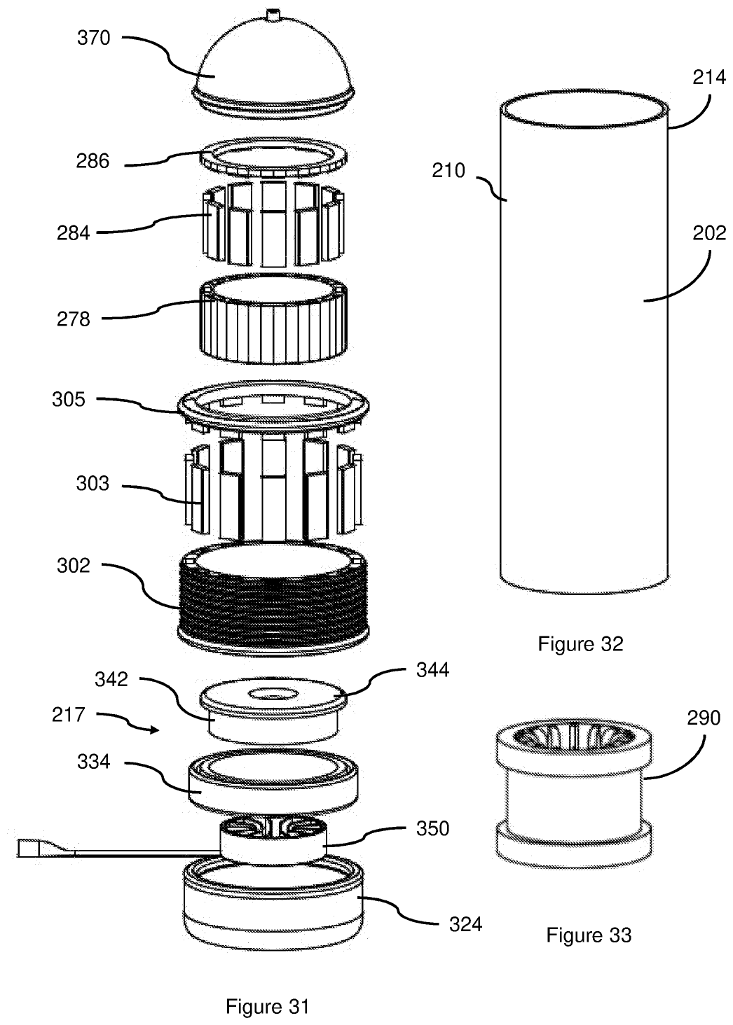

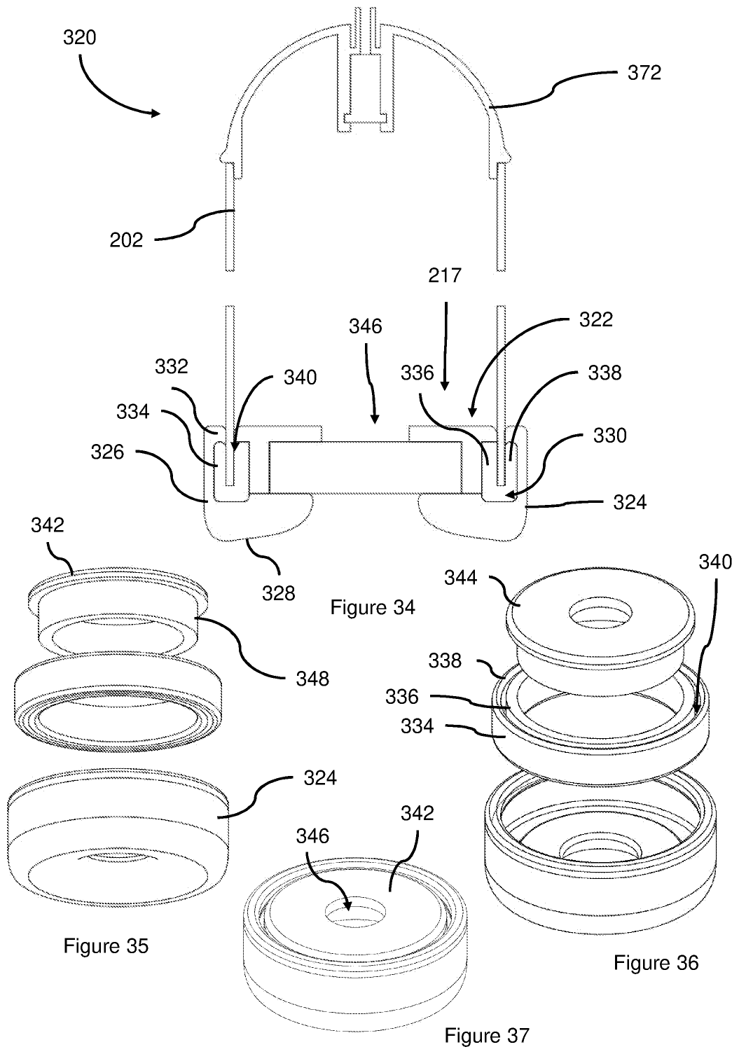

As can be seen in FIG. 31, the electrostimulation member 350 can be located within the wall 348 and interposed between the floor 328 and the end cap 342. It will be appreciated that there will be two electrostimulation members 350 for the members to function. These can be separated by a penile aperture member, of the type described herein, for example. Thus, the electrostimulation members 350 can form an electrostimulation insert for the penile aperture assembly 322.

It will readily be appreciated that the electrostimulation member 350 can be interchanged with the electrostimulation member 254 in either of the devices 200, 320. For example, as can be seen in FIG. 39, the proximal electrostimulation member 254.1 is positioned on the floor 328 and a proximal penile aperture member 360 is positioned on the member 254.1. The distal electrostimulation member 254.2 is positioned on the proximal penile aperture member 360. A distal penile aperture member 362 is positioned on the distal electrostimulation member 254.2. The aperture members 360, 362 are similar to the aperture members 236, 238. As shown in this drawing, reference numeral 356 indicates the lead in dotted lines to indicate that the electrostimulation members 254 can be replaced by the electrostimulation member 350. It will readily be appreciated that other forms of electrostimulation members could be positioned within a penile sealing arrangement 223, shown in FIG. 39.

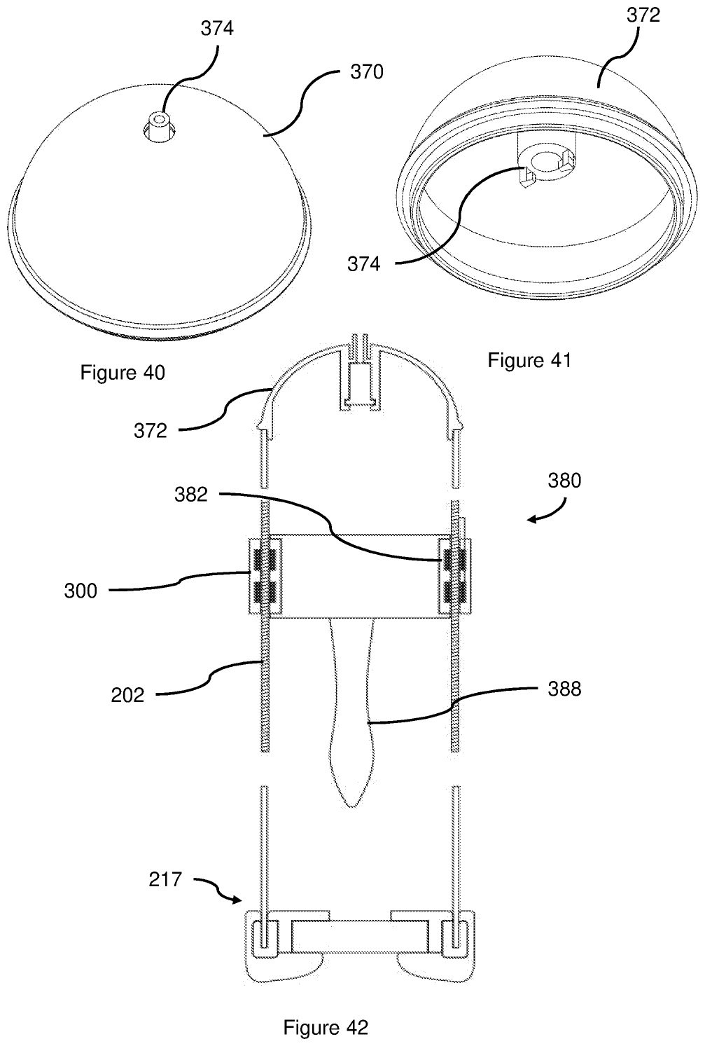

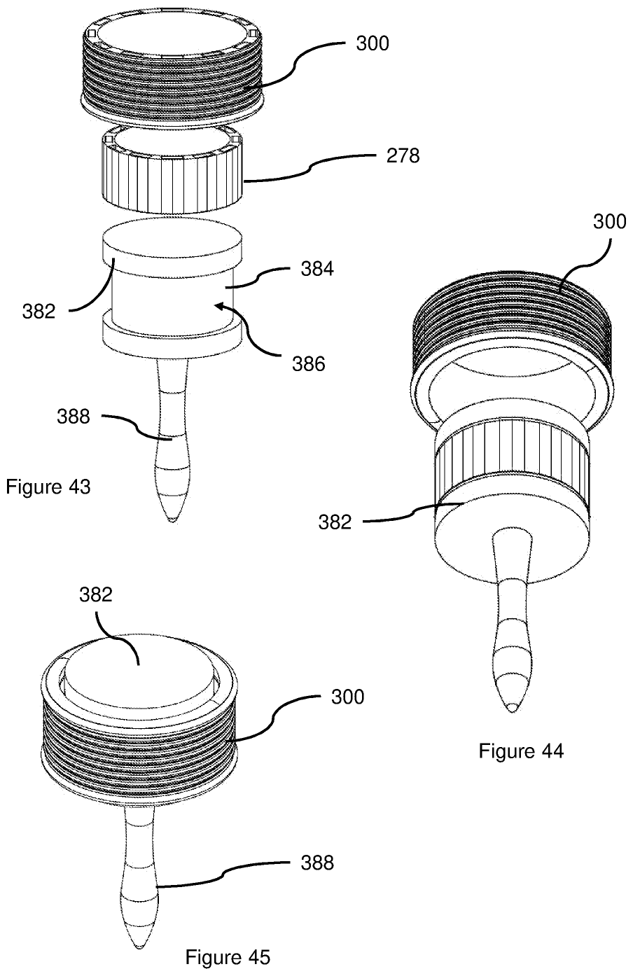

A closure 370 of the device 320 is shown in FIGS. 40 and 41. The closure 370 includes a cap 372 of a resiliently flexible material that can be press fitted onto a distal end of the tube 206. To that end, an edge of the cap 372 defines a shoulder 374 that can be brought into engagement with the distal end of the tube 206.

A vacuum pump connection port 374 is arranged in the cap 372 and is configured to engage a conduit of a vacuum pump, in a sealed manner, so that air can be extracted from the tube 206.

In FIG. 42, reference numeral 380 generally indicates a further embodiment of a stimulation and treatment device, in accordance with the invention. With reference to the preceding drawings, like reference numerals refer to like parts, unless otherwise specified.

The device 380 is particularly suited for vaginal stimulation. The device 380 includes a stimulation insert 382. The stimulation insert 382 includes a cylindrical body 384 of a resiliently flexible material, such as an elastomeric material. The cylindrical body 384 defines an annular recess 386. The inner slide 278 can be positioned in the recess 386 so that reciprocal movement of the external slider 300 results in reciprocal movement of the stimulation insert 382. The stimulation insert 382 includes an elongate stimulation member 388 that depends from the cylindrical body 384. The member 388 is configured so that it can move reciprocally in and out of the penile aperture assembly 322 as the external slider 300 is reciprocally driven or manipulated.

The stimulation member 388 can take any number of configurations. Furthermore, the device 380 can be provided with various stimulation inserts 382 that can be interchanged depending upon requirements.

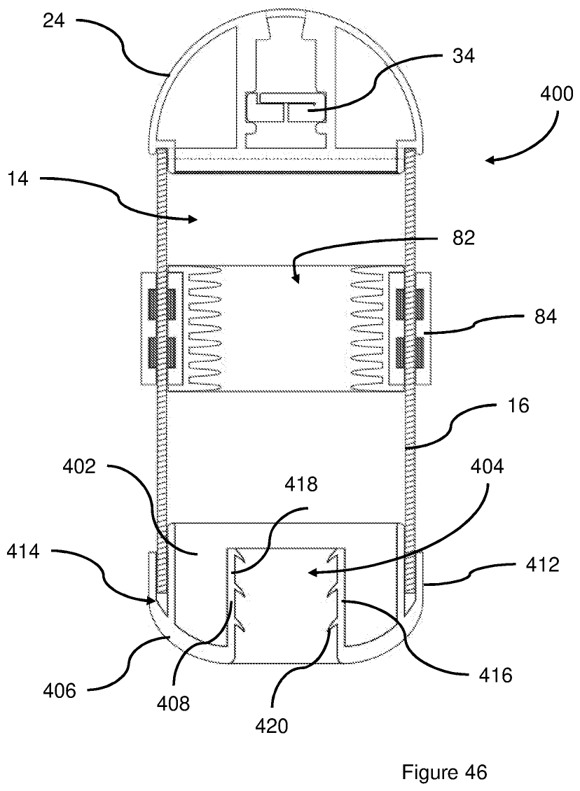

In FIG. 46, reference numeral 400 generally indicates another embodiment of a stimulation and treatment device, in accordance with the invention. With reference to the preceding drawings, like reference numerals refer to like parts, unless otherwise specified.

The device 400 includes a penile passage member in the form of a penile aperture assembly 402. The aperture member 402 defines a passage 404 in fluid communication with the chamber 14 and configured to engage the penis in a substantially airtight manner.

The aperture member 402 includes a fitting 406. The fitting 406 can be of an elastomeric material such as silicone or latex. The fitting 406 has a wall 408 that defines the passage 404. A peripheral sealing formation 412 is arranged on the wall 408 and is shaped to engage the proximal end 46 of the tube 16 in a detachable, sealed manner. The wall 408 defines a peripheral channel 414 in which the proximal end 46 of the tube 16 can be received.

The passage 404 is defined by an internal, cylindrical portion 416 of the wall 408. A series of annular sealing members 416 project from an internal surface 418 of the wall 408. The sealing members 416 are in the form of ribs or lips 420 that project proximally into the passage 410. The lips 420 can be tapered. Thus, when the penis is in the passage 410, ingress of air into the chamber 14 is inhibited as a result of radial pressure being exerted on the lips 420.

The components of the various embodiments of the stimulation and treatment device described above can readily be disassembled by hand for cleaning and/or replacement. Furthermore, it is to be appreciated that the components of the various embodiments of the stimulation and treatment device described above can readily be interchanged between the embodiments, where practical.

The stimulation and treatment device, of the various embodiments described above, is suited for penile stimulation and for the treatment of erectile dysfunction. The device can maintain an erection while the penis is stimulated through a reciprocal movement of the external slider. As a result, the device provides a means whereby a user can masturbate while the penis is maintained in an erect condition as a result of a vacuum in the chamber. As is known, a therapeutic method of treating erectile dysfunction requires the penis to be repeatedly engorged. However, without some form of stimulation, the erection is artificial. By allowing the user to self-stimulate, the device can result in the user ejaculating and/or experiencing an orgasm while maintaining the erection. This can help to train the user's ejaculatory system to achieve an erection and subsequent ejaculation and/or orgasm, ultimately, without the need for the device.

Repeated use of the device can result in a strengthening of the pelvic floor muscle. This can help to treat urinary continence.

Erectile dysfunction can be caused by various factors, including lifestyle factors and medical procedures, such as the partial or full removal of the prostate gland. Other factors can be psychological or neurological in nature. The device can help users to achieve an erection and then, subsequently, to ejaculate and/or experience an orgasm. Carried out repeatedly, this can be an effective treatment for erectile dysfunction, depending on the causes of the erectile dysfunction. Furthermore, this can be an effective release for users that are incapable of achieving a natural erection.

The appended claims are to be considered as incorporated into the above description.

The use of common reference numerals throughout the specification is not intended to be limiting and is solely for the purposes of clarity and ease of description.

Throughout the specification, including the claims, where the context permits, the term “comprising” and variants thereof such as “comprise” or “comprises” are to be interpreted as including the stated integer or integers without necessarily excluding any other integers.

In the specification, the word “distal” refers to an orientation that is away from a user, when compared to a “proximal” orientation which is closer to the user, in operation. Words indicating direction or orientation, such as “front”, “rear”, “back”, etc, are used for convenience. The inventor(s) envisages that various embodiments can be used in a non-operative configuration, such as when presented for sale. Thus, such words are to be regarded as illustrative in nature, and not as restrictive.

It is to be understood that the terminology employed above is for the purpose of description and should not be regarded as limiting. The described embodiments are intended to be illustrative of the invention, without limiting the scope thereof. The invention is capable of being practised with various modifications and additions as will readily occur to those skilled in the art.

1. A stimulation and treatment device, which comprises:
an elongate vessel that is closed at a distal end and that defines a vacuum chamber, the vessel including an air extraction passage in fluid communication with the vacuum chamber so that a pump can be connected to the vessel to extract air from the chamber via the air extraction passage;
a penile passage member arranged on a proximal end of the vessel, the penile passage member defining a penile passage through which a penis can extend into the vacuum chamber, the passage member being configured to engage a penis in a substantially sealed manner;
an internal slider arranged on an inner surface of the vessel and configured for sliding reciprocally within the vessel, the internal slider defining an opening through which the penis can extend and being configured to make contact with the penis to stimulate the penis during reciprocal movement of the internal slider; and
an external slider arranged on an outer surface of the vessel and capable of sliding reciprocal movement along the outside of the vessel, wherein
the internal and external sliders are magnetically attracted to each other so that the reciprocal manipulation of the external slider results in a corresponding reciprocal sliding of the internal slider, allowing the penis to be stimulated.

2. The device as claimed in claim 1, in which the elongate vessel includes a tube that is open at both ends and a closure that is detachably mounted on a distal end of the tube to close the distal end.

3. The device as claimed in claim 2, in which the closure is of an elastomeric material such as silicone and includes a seal that engages the distal end of the tube.

4. The device as claimed in claim 3, in which the closure defines the air extraction passage and a non-return valve assembly is positioned in the air extraction passage and is configured to permit the extraction of air from the chamber.

5. The device as claimed in claim 2, in which the closure includes a pump connection port to permit a pump to be connected to the closure for extracting air from the chamber.

6. The device as claimed in claim 1, in which the penile passage member includes a fitting that is configured to engage a proximal end of the tube in a substantially sealed, detachable manner, and a penis sealing arrangement positioned in the fitting and defining the penile passage so that the penis can be engaged in a substantially sealed manner by the sealing arrangement.

7. The device as claimed in claim 6, in which the penis sealing arrangement includes two rings, at least a portion of each ring being of a conductive material, conductive leads being connected to the conductive material to supply an electrical stimulating signal to the conductive material.

8. The device as claimed in claim 6, in which the penile passage member includes an electrostimulation insert that is positioned in the penis sealing arrangement to stimulate the penis, the electrostimulation insert being in conductive contact with the conductive material.

10-21. (canceled)

22. The device as claimed in claim 8, in which the electrostimulation insert includes two electrostimulation members positioned in spaced, aligned relationship, each electrostimulation member defining a passage through which a penis can extend such that the penis makes electrical contact with the electrostimulation members and each electrostimulation member is in contact with the conductive material of respective rings.

23. The device as claimed in claim 6, in which an electrostimulation insert is positioned in the penis sealing arrangement to make electrical contact with the penis, the electrostimulation insert being connectable to an electrical supply to receive an electrical stimulating signal.

24. The device as claimed in claim 23, in which the electrostimulation insert includes two electrostimulation members positioned in spaced, aligned relationship, each electrostimulation member defining a passage through which a penis can extend such that the penis makes electrical contact with the electrostimulation members and each electrostimulation member includes an electrical lead for connection to an electrical supply.

25. The device as claimed in claim 24, in which each electrostimulation member includes a body with a series of projections that extend into the passage from an inner surface of the body, the projections being positioned to make contact with the penis when the penis is received in the passage.

26. The device as claimed in claim 25, in which each electrostimulation member is of a resiliently flexible material.

27. The device as claimed in claim 24, in which a connector is arranged on the electrical lead, the connector being configured so that a transcutaneous electrical nerve stimulation machine can be connected to the lead.

28. The device as claimed in claim 1, in which the internal slider includes an annular inner slide and is dimensioned to make sliding contact with the inner surface of the tube.

29. The device as claimed in claim 28, in which an annular stimulation insert is mounted on the inner slide, the stimulation insert defining the opening through which the penis can extend.

30. The device as claimed in claim 29, in which the stimulation insert is in the form of a material suitable for reciprocal stimulation, such as a hypoallergenic, elastomeric material.

31. The device as claimed in claim 28, in which the inner slide includes a body with at least one magnet in or on the body.

32. The device as claimed in claim 1, in which the external slider includes an annular outer slide dimensioned to make sliding contact with the outer surface of the tube.

33. The device as claimed in claim 32, in which the outer slide includes a body with at least one magnet in the body.

34. An accessory for a stimulation and treatment device having an elongate vessel that is closed at a distal end and that defines a vacuum chamber, the vessel including an air extraction passage in fluid communication with the vacuum chamber so that a pump can be connected to the vessel to extract air from the chamber via the air extraction passage, an internal slider arranged on an inner surface of the vessel and configured for sliding reciprocally within the vessel, the internal slider defining an opening through which a penis can extend and being configured to make contact with the penis to stimulate the penis during reciprocal movement of the internal slider, and an external slider arranged on an outer surface of the vessel and capable of sliding reciprocal movement along the outside of the vessel, the internal and external sliders being magnetically attracted to each other so that reciprocal manipulation of the external slider results in a corresponding reciprocal sliding of the internal slider, allowing the penis to be stimulated, the accessory comprising:
a penile passage member that defines a penile passage through which a penis can extend, the penile passage member including:
a fitting that is configured to engage a proximal end of the elongate vessel in a substantially sealed, detachable manner; and
a penis sealing arrangement positioned in the fitting and defining the penile passage so that the penis can be engaged in a substantially sealed manner by the sealing arrangement.

35. An accessory for a stimulation and treatment device having an elongate vessel that is closed at a distal end and that defines a vacuum chamber, the vessel including an air extraction passage in fluid communication with the vacuum chamber so that a pump can be connected to the vessel to extract air from the chamber via the air extraction passage, a penile passage member arranged on a proximal end of the vessel, the penile passage member defining a penile passage through which a penis can extend into the vacuum chamber, the passage member being configured to engage a penis in a substantially sealed manner, the accessory comprising:
an internal slider configured for arrangement on an inner surface of the vessel and configured for sliding reciprocally within the vessel, the internal slider defining an opening through which a penis can extend and being configured to make contact with the penis to stimulate the penis during reciprocal movement of the internal slider; and
an external slider configured for arrangement on an outer surface of the vessel and capable of sliding reciprocal movement along the outside of the vessel, the internal and external sliders being magnetically attracted to each other so that reciprocal manipulation of the external slider results in a corresponding reciprocal sliding of the internal slider, allowing the penis to be stimulated.
